(19) United States
(12) Patent Application Publication
(10) Pub. No.: US 2012/0197158 A1
(43) Pub. Date: Aug. 2, 2012

(54) Handheld analysis device

(76) Inventor: Wolfgang Roedel, Heidelberg (DE)

(21) Appl. No.: 13/196,970

(22) Filed: Aug. 3, 2011

Related U.S. Application Data
(63) Continuation of application No. PCT/EP2010/000994, filed on Feb. 17, 2010.

(30) Foreign Application Priority Data
Feb. 18, 2009 (EP) 09002281.5

(51) Int. Cl. A61B 5/151

(52) U.S. Cl. 600/583

(57) ABSTRACT

A handheld medical device for body fluid analysis such as to measure an analyte concentration, for example glucose, in a body fluid such as blood or interstitial fluid by a patient or caregiver. The device comprises a mechanical assembly having a transport unit for consumable analytical elements, a display unit, a sensor for analyzing a body fluid sample obtained by a lancet prick, a memory for storing a series of measurement results, a first actuating element, the actuation of which transfers the device into a first active state, activates the mechanical assembly and prepares a measurement. In the first active state, a measurement is started by the actuation of one of the actuating elements, and a second actuating element, the actuation of which transfers the device into a second active state. The display unit is switched on by the actuation of the second actuating element, without activating the mechanical assembly.

### REFERENCE

This application is a continuation of PCT/EP2010/000994 filed Feb. 17, 2010 that is based on and claims priority to European Patent Application No. EP 09002281.5 filed Feb. 18, 2009, which are hereby incorporated by reference.

### FIELD

This disclosure relates to a handheld medical device for body fluid analysis which can be used to measure an analyte concentration, such as glucose, in a body fluid such as blood or interstitial fluid.

### BACKGROUND

Handheld analysis devices are used by patients, such as persons with diabetes, who can be required to check their blood glucose level several times a day by obtaining a body fluid sample, in general blood or interstitial fluid, obtained from a small puncture wound. A sensor contained in the handheld analysis device can be used to measure an analyte concentration, for example the glucose concentration, of a body fluid sample obtained by a lancet prick. An example of an analysis device integrated into a wrist watch is shown in US 2006/0178600 A1, Blood analyzer and pricking device for use in blood analysis by Kennedy et al. (Aug. 10, 2006).

It is a constant goal in the development of handheld analysis devices to enable users the easiest handling possible and allow for a measurement to be conducted quickly.

### SUMMARY

A handheld medical device is disclosed for measuring a body fluid sample such as blood or interstitial fluid with improved comfort. The device comprises a mechanical assembly having a transport unit for consumable analytical elements, a display unit, a sensor for analyzing a body fluid sample obtained by a lancet prick, a memory for storing a series of measurement results, a first actuating element, the actuation of which transfers the device into a first active state, activates the mechanical assembly and prepares a measurement. In the first active state, a measurement is started by the actuation of one of the actuating elements, and a second actuating element, the actuation of which transfers the device into a second active state. The display unit is switched on by the actuation of the second actuating element, without activating the mechanical assembly.

Some embodiments of the handheld analysis device can be transferred into a first active state by means of a first actuating element. In the first active state, a mechanical assembly comprising a transport unit for consumable analytical elements, for example test elements and/or lancets, is activated and a measurement prepared. In the first active state, a measurement can be started by actuating one of the actuating elements of the handheld analysis device. Some embodiments, additionally have a second active state, in which the display unit is switched on without activating the mechanical assembly. Results of previous measurements can be displayed in this second active state, for example to allow a user to check earlier treatment progress.

When the mechanical assembly is activated, at least one component of the assembly is moved, for example a test element and/or a lancet are removed from a supply. In the case of simple embodiments of the handheld analysis device, the user must exert the force required for this, while in more complex embodiments of the handheld analysis devices, the mechanical assembly of which contains an electric motor, a load is applied to an electric energy storage means, typically a battery. In some embodiments, this expenditure can only take place when a user in fact wants to carry out a measurement. However, if a user does not want to carry out a measurement, but for example only wants to view a series of previous measurements or change a device setting, for example set a clock integrated in the device, the expenditure associated with the preparation of a measurement can be omitted which would omit movement of mechanical components.

Some embodiments of the handheld analysis device can be designed purely as a measuring device, which together with a separate lancing device forms a measuring system. However, in some embodiments a lancing device is integrated in a handheld analysis device. In such embodiments, the mechanical assembly additionally comprises a lancing drive so as to cause the lancets to perform a lancing movement.

The consumable elements can be, for example, test elements containing detection reagents for determining a concentration, notably for photochemical or electrochemical concentration determination, or they can be lancets. The consumable elements can in particular also be lancets comprising integrated test elements. Lancets comprising a test region can be used as test elements, for example. Such a test region can be, for example, a cuvette for reagent-free analysis, or a test field containing detection reagents. Test fields containing detection reagents for the photometrical or electrochemical concentration determination have been used in commercially available test strips for determining the glucose concentration for decades. Corresponding test fields can be glued onto a lancet. Such a lancet may comprise a channel, for example in the form of a groove, which causes fluid to be transported to a test field by means of capillary forces. However, it is also possible to use lancets and separate test elements as consumable elements in a handheld analysis device according to the invention. Such test elements can take up a body fluid sample from a lancet, which for this purpose is guided by the mechanical assembly of the handheld analysis device to the test element. It is also possible for separate test elements to take up a body fluid sample directly from a previously generated puncture wound.

The consumable element supply for a handheld analysis device, in some embodiments, can be designed as a magazine that can be inserted in the device. Such a magazine can contain test elements and/or lancets in separate magazine chambers. However, it is also possible to design such a magazine as a cassette comprising a wound-up carrier tape, which carries test elements and/or lancets or lancets comprising integrated test elements. For a handheld analysis device that is a measuring device and a lancing device, an internal supply of test elements and lancets. However, it is also possible, for example, to provide only a supply of lancets in the device and to individually introduce test elements into the device for a measurement after a puncture.

A measurement can be prepared, for example, by removing a test element and/or a lancet from a supply by charging a mechanical energy storage means of a lancing drive, or by coupling a lancing drive to a lancet. In some embodiments, all these steps, or only a part of these steps, can be carried out to prepare a measurement. It is also possible, for example, to remove a test element or a lancet from a supply after a measurement is completed and to bring it into a usage position, in which the test element or the lancet remains until the next measurement. However, test elements and/or lancets are preferably not removed from a magazine in the first active state of the device until a measurement is prepared, because they are protected best from harmful effects of the environment in the magazine.

The mechanical assembly can be deactivated after activation by actuating the first actuating elements. Some embodiments can automatically deactivate following a predefined time period if no measurement was carried out by then. The predefined time period can be rigidly specified by the device manufacturer and, for example, range between half a minute and ten minutes, and more particularly between two and five minutes.

When a measurement is prepared and the mechanical assembly is activated, it is possible that a user may not want to carry out a measurement, for example because the activation was done inadvertently, or the user has changed her mind. When the mechanical assembly is activated, for example a test field or a lancet can be removed from a sterile packaging. After some time, the function of the test field or the sterility of the lancet may not be guaranteed any longer. By deactivating the mechanical assembly, it can be ensured that test fields and/or lancets are only used promptly after a measurement is prepared. If no measurement takes place within the predetermined time, the test field and/or the lancet is advanced, at the latest upon renewed activation of the mechanical assembly, for example in a space in the device that is provided for used test fields and/or lancets.

The handling of an unused test field causes no particular difficulties when deactivating the mechanical assembly. Until it is activated again, the test field can remain in its position and is simply advanced when it is activated again. Because of the lancing drive of the device, however, additional measures may be called for in the case of a lancet, in particular when the activation of the mechanical assembly is associated therewith, so that a mechanical energy storage means of the lancing drive is charged, for example a drive spring is tensioned. In such a case, the mechanical energy storage means is preferably discharged when the mechanical assembly is deactivated. This is preferably done by triggering a puncture.

In some embodiments, the first actuating element can be, for example, a cover of a device opening on which a sample is taken up. When the device opening is exposed, the mechanical assembly is activated. When the device opening is closed again without a puncture being triggered beforehand, a puncture can be triggered in this way. The actuating element, which is to say the cover, preferably comprises a depression for a lancet on the inner side.

However, it is also possible for the energy storage means to be discharged again without triggering a puncture. While this necessitates a slightly more complex mechanism of the lancing drive, automatic activation can be provided. However, it is also possible for the device to automatically deactivate following a predefined time period if no measurement was carried out by then, and for the device opening to close, whereby the actuating element is automatically moved. After the device opening is closed, a puncture can be triggered without risk of injury to the user.

### BRIEF DESCRIPTION OF THE DRAWINGS

Further details and advantages of the invention will be described based on an embodiment with reference to the attached drawings.

FIG. 1 shows a schematic illustration of a portable blood glucose measuring device embodiment; and

FIG. 2 shows a schematic illustration of the measuring device shown in FIG. 1 comprising a cut-open housing embodiment.

### DETAILED DESCRIPTION

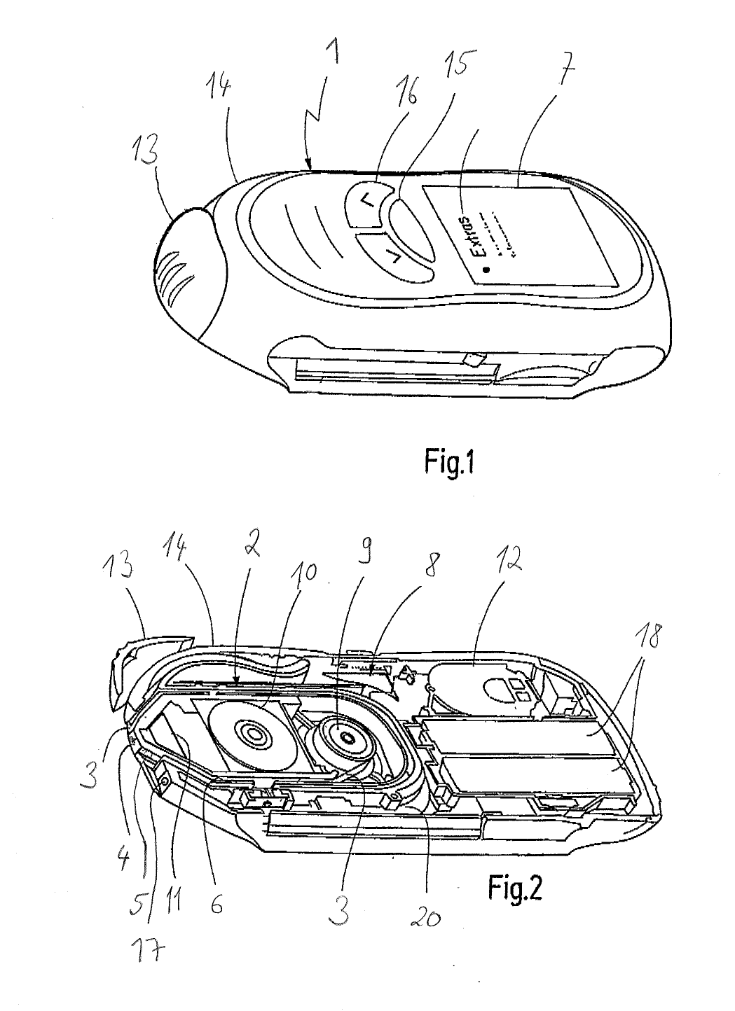

The handheld analysis device 1 embodiment shown in FIGS. 1 and 2 enables a user to determine the glucose concentration of a body fluid sample in a substantially automatic measuring process. A magazine 2 comprising a test element supply can be inserted in a receiving compartment of the device 1. For this purpose, a housing cover 20 on the bottom can be removed. In the embodiment shown, the magazine 2 is a tape cassette, which carries lancets 4 and test elements 5 on a carrier tape 3. Instead of such a cassette, it is also possible, for example, to use a magazine comprising individual magazine chambers.

A body fluid sample that was obtained by a lancet prick can be analyzed by a sensor 6, and an analyte concentration, for example the glucose concentration or the lactate concentration, can be determined. Measurement results can be displayed with a display unit 7 of the device 1, for example a liquid crystal display, and stored in a memory of the device 1, which is not shown.

Together with a control and evaluation unit 8, such as a microprocessor, and the display unit 7, the memory forms an electronic assembly, which enables, for example, the evaluation and representation of evaluation results of a series of measurement results, which can be stored together with the date and time of the underlying measurement in the memory.

The handheld analysis device 1 additionally contains a mechanical assembly, which comprises at least one transport unit 9 for test elements. In the illustrated embodiment, the transport unit 9 causes the carrier tape 3 to be wound on a winding reel of the cassette 2 and to be unwound from a supply reel 10, which is to say it causes tape to be transported. In the handheld analysis device, which like the illustrated embodiment is a lancing device at the same time, the mechanical assembly additionally contains a lancing drive 11 so as to cause the lancets 4 to perform a piercing movement. The lancing drive 11 is like an additional transport unit, because a lancet is moved or transported during a puncture in the puncture direction. The mechanical assembly can also contain an electric motor 12. In general, however, the force supplied by the electric motor to the transport unit 9 and the lancing drive 11 can also be applied by a user.

The lancing drive 11 can contain a mechanical energy storage, for example a spring element made of synthetic material or metal, which releases the kinetic energy required for a fast lancet prick as needed. Such a mechanical energy storage means can be charged by the electric motor 12 of the handheld analysis device 1, for example by tensioning a spring. The lancing drive 11 can, for example, cause a puncture movement in the manner described in WO 2008/083844 A1, Pricking device by Konya (17 Jul. 2008).

The handheld analysis device 1 embodiment has a first actuating element 13, the actuation of which transfers the device 1 into a first active state, activates the mechanical assembly and prepares a measurement. The first actuating element 13 is preferably designed as a cover of a device opening on which a sample is taken up. The first actuating element 13 is thus actuated by being moved out of a starting position, in which it covers the device opening, into a different position, in which the device opening is exposed.

An integrated system can be desirable, which is to say a handheld device having lancing and measuring functions, when the time between switching on the device and displaying the measuring result is very short. On the other hand, the display of the result must be reliable. Prior to a puncture, the function of the device and of the consumable material inserted therein should therefore be checked. The corresponding tests can be carried out automatically as soon as the device is transferred into the first active state.

To prepare and carry out a measurement, for example, some or all of the following steps can be carried out:

exposing a device opening for taking up a sample;

display test;

checking the coding and data of consumables, for example by reading data of a magazine and checking it by calculating a test sum;

checking the measuring unit, for example to determine potential defects or contamination, in particular of an optical path for photometric measurements;

measuring a test field in an empty state prior to taking up a sample;

measuring the device temperature and/or humidity;

safety query to detect accidental activation of the device without intending a measurement;

activating the mechanical assembly, notably moving a lancet into the lancing position thereof and tensioning a lancing drive;

triggering a puncture;

taking up a sample;

carrying out a measurement;

calculating a concentration value from a measurement signal;

displaying the measurement result; and

deactivating the mechanical assembly and the device.

The first actuating element 13 can be fastened pivotably or displaceably on the device housing 14, for example. The first actuating element 13 can be designed, for example, as a protective cap and protect the interior of the device 1 from contamination. By designing the first actuating element 13 as a cover for the device opening provided for taking up a sample, user comfort is improved, because the mechanical assembly is only activated when a user indeed plans to carry out a measurement, since the user will generally expose the housing opening only then. The actuating element 13 designed as a cover so the user does not perceive the time between the movement of the cover and placing a finger on the housing opening as a waiting period, yet generally sufficient time remains between the movement of the cover and the placement of a finger on the housing opening to prepare the device for a measurement. A user thus may gain the impression that a measurement can be started “immediately”.

When a user does not intend to carry out a measurement, and instead, for example, only wants to modify device settings or have existing measurement results displayed, he can transfer the device 1 into a second active state by actuating a second actuating element 15, which can be designed as a button, for example, and switch the display unit 7 on without activating the mechanical assembly. In the illustrated embodiment, further actuating elements 16 are disposed on the front of the handheld device 1 carrying the display unit 7. These can be used, for example, to select commands from a control menu displayed by the display unit 7. The display unit 7 can be designed as a sensor field and form an additional actuating element. The device opening provided for taking up the sample thus remains closed in the illustrated embodiment when the device is transferred into the second active state by the actuation of the second actuating element 15.

When the handheld analysis device 1 embodiment is in the first active state, a measurement can be started by actuating one of the actuating elements, which is to say a lancet prick can be triggered in the illustrated embodiment. For this purpose, a third actuating element 17 can be provided, which is disposed on the device opening. The third actuating element 17 is actuated by placing a body part on which a puncture wound is to be generated against the device opening. The third actuating element 17 can contain a temperature and/or pressure sensor, which establishes not only whether a body part has been placed thereon, but additionally verifies that the conditions for sampling are favorable, for example the pressure with which the body part rests on the device opening, or that the temperature of the body part is within a predefined range. The handheld analysis device 1 embodiment thus comprises a plurality of actuating elements 13, 15, 16, 17 for operating the handheld analysis device 1.

In the starting position, in which it covers the device opening, the first actuating element 13 can cover the third actuating element 17, so that the same is protected in the inactive state of the device 1.

After a measurement is concluded, the handheld analysis device 1 automatically, which is to say without the involvement of a user, can transition into the second active state, so that a user can use additional device functions, for example view evaluations of a series of measurements and thus check the treatment progress, or modify device settings, for example adjust wake-up or reminder functions to other times. From the second active state, the device can transition into an inactive state, in which the display unit is switched off. From the second active state, the device can be transferred into the inactive state, which is to say switched off, by actuating an actuating element provided for this purpose. The device also preferably transitions automatically from the second active state into the inactive state when no actuating element was actuated during a predefined time period. In this case, it is assumed that the user has forgotten to switch the device off. So as to avoid unnecessary power consumption, the device then switches off automatically. The predefined time period can be rigidly specified by the device manufacturer and range, for example, between half a minute and two minutes.

The electric motor 12 and the electronic assembly comprising the display unit 7 and the control and evaluation means 8 are powered by a source such as one or more batteries 18, which can be inserted in a compartment of the device 1 provided for this purpose and replaced if necessary.

When a user does not want to carry out a measurement after the mechanical assembly is activated, for example because the first actuating element 13 was actuated accidentally, the mechanical assembly can be deactivated by actuating the first actuating element 13 again. By actuating the first actuating element 13 again, the device opening provided for taking up a sample is closed again. As a result, the mechanical assembly is deactivated and the device transitions into the second active state. A puncture is triggered during this deactivation of the mechanical assembly. A depression may be provided on the inner side of the first actuating element 13, which is to say the cover of the device opening, so that the lancet can perform the usual pricking motion.

Thus, embodiments of the handheld analysis device are disclosed. One skilled in the art will appreciate that the teachings can be practiced with embodiments other than those disclosed. The disclosed embodiments are presented for purposes of illustration and not limitation, and the invention is only limited by the claims that follow.

What is claimed is:

1. A handheld analysis device, comprising:
a mechanical assembly, which comprises a transport unit for consumable analytical elements;
a display unit;
a sensor for analyzing a body fluid sample obtained by a lancet prick;
a memory for storing a series of measurement results;
a first actuating element, the actuation of which transfers the device into a first active state, activates the mechanical assembly and prepares a measurement, with the measurement being started in the first active state by actuating one of the actuating elements; and,
a second actuating element, the actuation of which transfers the device into a second active state;
wherein the display unit is switched on by the actuation of the second actuating element, without activating the mechanical assembly.

2. The handheld analysis device as in claim 1 wherein the first actuating element is a cover for a device opening on which the body fluid sample is taken up.

3. The handheld analysis device as in claim 2 wherein the cover can be pivoted.

4. The handheld analysis device as in claim 2 wherein a third actuating element is provided for triggering a measurement, the third actuating element being disposed on a device opening and configured to be actuated by placing a body part on which a puncture wound is to be generated against the device opening.

5. The handheld analysis device as in claim 4 wherein a starting position, the first actuating element covers the third actuating element, and the first actuating element covers the device opening.

6. The handheld analysis device as in claim 1 wherein the mechanical assembly comprises a lancing drive so as to cause the lancets to perform a piercing movement.

7. The handheld analysis device as in claim 6 wherein a measurement is prepared by charging a mechanical energy storage means of the lancing drive.

8. The handheld analysis device as in claim 7 wherein a measurement is prepared by coupling the lancing drive to a lancet.

9. The handheld analysis device as in claim 1 wherein the consumable elements are test elements and/or lancets.

10. The handheld analysis device as in claim 1 wherein the mechanical assembly comprises an electric motor.

11. The handheld analysis device as in claim 1 wherein a measurement is prepared in that an consumable element is removed by the transport unit from an consumable element supply.

12. The handheld analysis device as in claim 1 wherein the display unit is switched on by actuating the first actuating element.

13. The handheld analysis device as in claim 1 wherein device settings can be modified in the second active state.

14. The handheld analysis device as in claim 1 wherein measurement results can be displayed by the display unit in the second active state.

15. The handheld analysis device as in claim 1 wherein it transitions from the second active state into an inactive state, in which the display unit is switched off.

16. The handheld analysis device as in claim 15 wherein it automatically transitions from the second active state into the inactive state when no actuating element was actuated during a predefined time period.

17. The handheld analysis device as in claim 1 wherein the mechanical assembly can be deactivated by actuating the first actuating element.

18. The handheld analysis device as in claim 1 further comprising, a magazine that can be inserted in the device, the magazine containing a consumable element supply.

19. A handheld analysis device, comprising:
a mechanical assembly, which comprises a transport unit for consumable analytical elements;
a display unit;
a sensor for analyzing a body fluid sample obtained by a lancet prick;
a memory for storing a series of measurement results;
a means for first actuating to transfer the device into a first active state, activates the mechanical assembly and prepares a measurement, with a measurement being started in the first active state by actuating one of the actuating elements; and,
a means for second actuating to transfer the device into a second active state that switches on the display unit without activating the mechanical assembly.
